(19) United States
(12) Patent Application Publication
(10) Pub. No.: US 2003/0050249 A1
(43) Pub. Date: Mar. 13, 2003

(54) Complexes of alpha-6 integrin subunits with small peptides and methods for treating indications resulting from modulation of integrin-mediated responses by altering signal transduction

(76) Inventors: James A. Clagett, Snohomish, WA (US); John Lipani, Mountain Hills, AZ (US); Craig Robert Palmer, San Francisco, CA (US)

Correspondence Address: Dike, Bronstein, Roberts & Cushman, Intellectual Property Practice Group, Edwards & Angell, LLP, 101 Federal Street, Boston, MA, 02209, US

(21) Appl. No.: 09/863,837

(22) Filed: May 23, 2001

Related U.S. Application Data
(60) Provisional application No. 60/206,397, filed on May 23, 2000.

(51) Int. Cl. A61K 38/07; A61K 31/573; C07K 14/705

(52) U.S. Cl. 514/18; 514/171; 530/350

(57) ABSTRACT

A method for modulating an alpha 6 subunit containing integrin-mediated signal transduction is described. The method involves contacting a cell with an effective integrin modulating amount of an alpha 6 subunit containing integrin-mediated signal transduction pathway modification agent. Preferred agents are peptides having the formula f-Met-Leu-X, wherein X is selected from the group consisting of Tyr, Tyr-Phe, Phe-Phe and Phe-Tyr.

### FIELD OF THE INVENTION

This invention relates to N-formyl peptides and cell surface receptors, and particularly to complexes of alpha-6 integrin subunits with an α6 subunit containing integrin-mediated signal transduction pathway modification agent, preferably certain N-formyl peptides. The invention further relates to methods for treating indications resulting from integrin-mediated responses, and particularly to methods for modulating integrin-mediated signal transduction resulting from cell stimulation by pro-inflammatory agents.

### BACKGROUND OF THE INVENTION

N-formylmethionyl Peptides

The human body has evolved to develop defense mechanisms to bacterial infections by using bacterially generated N-formylmethionyl peptides as chemoattractants for phagocytes, in particular, neutrophils and monocytes. Of the N-formyl peptides, f-Met-Leu-Phe (fMLP) was identified as the most potent in its ability to recruit phagocytes and to stimulate release of lysosomal enzymes by neutrophils (Showell et al., J. Exp. Med. 143:1154-1169, 1976). Synthetic tetrapeptides, particularly f-Met-Ile-Phe-Leu and f-Met-Leu-Phe-Ile, have also subsequently been shown to evoke neutrophil responses (Rot et al., Proc. Natl. Acad. Sci. USA 84:7967-7971, 1987). The potency of these peptides to recruit phagocytes and to stimulate release of lysosomal enzymes were initially ascribed to: (1) a formyl group at the N-terminus, (2) a methionine side chain, and (3) leucine and phenylalanine side chains.

The most well-studied N-formyl peptide is f-Met-Leu-Phe (FMLP, fMLP or MLF). However, more potent peptides have been characterized with N-formyl peptide receptors (FPR) on rabbit neutrophils in vitro. In particular, fMet-Leu-Phe-Phe, fMet-Leu-Phe-NHBzl (fMet-Leu-Phe benzylamide), and fNle-Leu-Phe-Tyr (N-formyl-L-norleucyl-Leu-Phe-Tyr) (Kermode et al., Biochem. J., 276: 715-723, 1991) showed both maximal migration (on the order of 20-35 μm) and degranulation (on the order of ED₅₀ of 10⁻¹⁰ to 10⁻¹¹). More recent reports suggest that nonformylated peptides may also bind to FPR and can act as potent activators of neutrophil function. For example, Met-Met-Trp-Leu-Leu is a potent pentapeptide and is comparable in neutrophil function activity to FMLP (Chen et al., J. Biol. Chem. 270: 23398-23401, 1995). Conversion of the pentapeptide to an N-formylated form boosted its potency 100-500 fold, demonstrating that N-formylation still plays a significant role in the potency of a peptide, although bioactivity does not appear to be strictly determined by N-formylation.

Other modifications to peptides have shown that some peptides can be converted to potent agonists for FPR (Derian et al., Biochemistry 35: 1265-1269, 1996; Higgins et al., J. Med. Chem. 39: 1013-1017, 1996). Such modifications include urea substitution of the amino terminal group and carbamate modifications. Furthermore, alteration of amino acid composition of the MLF peptide also has been shown to convert agonists to antagonists of FPR, as determined by superoxide anion release and neutrophil adhesion to vascular endothelium.

Integrins

The integrins are transmembrane proteins found on virtually every cell type. Their intracellular domain binds to the cytoskeleton while their extracellular domain can bind to a variety of ligands, including collagens, laminin, von Willebrand factor, thrombospondin and fibronectin. Thus, integrins serve as a link between the inside and the outside of the cell and can participate in ‘inside-out’ and ‘outside-in’ signal transduction. Integrin-mediated signal transduction is involved in the initiation of actin cytoskeleton organization and polymerization, cellular responses to the extracellular matrix (ECM) proteins and cellular responses to growth factors.

Inactive (basal level) integrins exhibit low affinity for ligand, but upon activation via phospholipase C (PLC), phosphatidylinositol (PI3) and the Rho family of GTPases to initiate “inside-out” or “outside-in” signaling, integrins participate in high-affinity ligand binding, consequently regulating cell growth and/or apoptosis, spreading, migration and adherence to various tissues. Ca++ flux initiated by such tyrosine or serine/threonine kinases, as mediated by G-proteins, is normally associated with the initiation of the signals, which effect agonist or inhibitory effects with integrin-mediated cellular activity (Jennings et al., Cell. Mol. Life Sci. 54:514-526, 1998).

The integrins are transmemebrane glycoproteins that have been identified as having sixteen α and eight β subunits. An integrin cell surface receptor is formed by a noncovalent interaction between an α and a β subunit to form a heterodimer; 22 such heterodimers have currently been identified. Based upon the various combinations of these 22 heterodimers, more than 170 classifications of integrins have been identified.

The α subunits are composed of a transmembrane domain, a short cytoplasmic tail and a large extra cellular domain (˜1,000 amino acids). The extracellular domain is made of seven 60 amino acid tandem repeats that are highly homologous to the divalent cation binding sites found in many calcium binding proteins. The β subunits are smaller than the a subunits but are also transmembrane proteins with a cytoplasmic tail and an extracellular domain which may bind divalent cations. For both the α and the β subunits, the amino terminus forms the extracellular domain while the carboxy terminus forms the cytoplasmic domain.

Active binding sites have been mapped in the alpha subunits wherein they bind ligands with specific amino acid sequences which are believed to be mandatory for integrin regulation. Two phenylalanine residues and the terminal arginine residue are believed to be mandatory in integrin affinity regulation (Shattil et al., J. Biol. Chem. 271:269-271, 1996).

Integrins play a central role in the inflammatory response. Activation of neutrophils is mediated by N-formyl peptides generated at the site of infection, injury or disease leading to neutrophil accumulation at this site. N-formyl peptides upregulate L-selectin on neutrophils and direct rolling of neutrophils along the endothelium, followed by upregulation of integrins on the surface of neutrophils. Integrins mediate cell-cell and cell-extracellular matrix interactions and bind to laminin, fibronectin, vitronectin, as well as to ICAM (intracellular cell adhesion molecule) and VCAM (vascular cell adhesion molecule) found on the endothelium. Upon binding of the integrins to ICAM and VCAM, a signal is transduced to the interior of the neutrophil through interactions with the cytoskeleton. Neutrophils then shed L-selectin and begin to spread along the endothelium. Upregulation of E-selectin and ICAM-1 on the surface of endothelial cells then mediate the migration of neutrophils across the endothelium (Luscinskas et al., J. Immunol. 146: 1617-1625, 1991). Upon crossing the endothelial barrier, neutrophils migrate toward the site of inflammation by sensing a concentration gradient of the N-formyl peptide. Upon reaching their destination, which contains a high concentration of the peptide, neutrophils unleash their anti-microbial actions.

Integrin regulation has been repeatedly found to be involved in cancer metastasis, dictating the anchorage-independent growth, survival and motility of tumor cells, as well as promoting tumor cell invasion and angiogenesis (Clezardin, Cell Mol. Life Sci. 54: 541-548, 1998).

Integrins are now implicated in thrombosis, atherosclerosis and coronary heart disease (CHD) through their regulation of platelet spreading and aggregation, as well as involvement with the thrombospondin receptor (Lindner et al., J. Biol. Chem. 274: 8554-8560, 1999).

Given the crucial role integrins serve in regulating a variety of major disease indications, extensive research efforts have recently been expended in the attempt to develop therapeutics which might regulate integrin function. While much of this effort has been placed upon monoclonal antibodies (mABs), an extensive search of a variety of natural products (e.g., snake venoms, fungal wortmannin, etc.) has been undertaken with an effort to develop a therapeutic modality. Such agents might involve antagonist action with key integrins in effecting anti-inflammatory or anti-neoplastic outcomes. To date, the most promising therapeutic results have been found with mABs involved in regulation of ICAM and VCAM for cardiac and transplantation uses. However, because of the exquisite specificity of mABs, their wider utility to treat a variety of inflammatory diseases has yet to be achieved. Naturally occurring small peptides that may provide a more effective bridge to whole families of integrins through commonality of α and β-chain expression has recently gained increased impetus in the search for integrin-based therapeutics.

VLA-6 is a glycosylated integrin receptor composed of the α₆β₁ subunits. VLA-6 functions as a laminin receptor in platelets, endothelial cells, epithelial cells, fibroblasts, T lymphocytes, neutrophils, monocytes and thymocytes. The binding of VLA-6 to laminin appears to be monospecific. The alpha 6 subunit can also associate with the beta 4 subunit on epithelial cells.

The expression of the alpha 6 integrin subunit is associated with transformation and tumor progression. Increased levels of alpha 6 expression are associated with tumors of the head and neck, bladder and lung cancer and colon carcinoma (Varner, J. A. et al., Curr. Opin. Cell Biol., 8: 724-730, 1996).

Signal Transduction

Stimulated neutrophils rapidly activate respiratory burst oxidase, which catalyzes the generation of the superoxide radical O₂−. The superoxide radical reacts with other molecules to produce hydrogen peroxides and hypochlorous acid, both of which are highly reactive agents and are therefore effective in interfering with microbial functions. Degranulation is also an effective means for destroying foreign microbes. However, degranulation can also damage host tissue. Phagocytosis is another mechanism by which neutrophils eliminate foreign microbes. Many of these functions are stimulated via the G-protein, using phospholipases as second messengers, three of which have been characterized.

The phospholipase C, PLC_{β2}, generates two second messengers, 1,4,5-inositol triphosphate (IP₃) and diacylglycerol (DG). The βγ subunits of the G-protein generated during activation of the FPR activate PLC_{β2}. IP₃ binds to certain calcium channels to stimulate the release of calcium from intracellular storage, resulting in an increase in the cytosolic concentration of calcium that is observed during stimulation by chemoattractants. DG, in concert with released calcium, activates protein kinase C (PKC). G-protein activated PLC kinase has recently been reported in the literature (Beaven, et al, J. of Immunology 160:5136-5144, 1998) as a major pathway for mast cell degranulation in rat peritoneal cells in vitro, associated with Ca²⁺ increases.

Phospholipase A₂ (PLA₂) generates arachidonic acid from the phospholipids of the inner face of the plasma membrane. Arachidonic acid provides the precursors for the inflammatory mediators such as leukotrienes and prostaglandins. PLA₂ is activated upon phosphorylation by the mitogen-activated protein (MAP) kinase.

The third phospholipase is phospholipase D (PLD), which generates phosphatidic acid and choline from phosphatidylcholine. Phosphatidic acid may be involved in activation of respiratory burst oxidase in addition to playing a role in the production of DG, which activates PKC. However, activation of PLD requires calcium, and FMLP cannot stimulate PLD in calcium-depleted cells (Kessels et al., J. Biol. Chem. 266: 23152-23156, 1991). In addition, it appears that the G-protein Arf and G-protein Rho regulate PLD activity (Brown et al., Cell 75: 1137-1144, 1993; Cockcroft et al., Science 263: 523-526, 1994; Singer et al., J. Biol. Chem. 270: 14944-14950, 1995).

Phosphorylation of a variety of substrate proteins by protein kinases is another way in which FPR and integrins transduce extracellular signals, such as binding of fMLP, to the interior of the cell. Some of the major protein kinases involved in FPR and integrin signaling are discussed below.

As discussed above, PKC is activated by DG, which is generated by PLC. PKC act to phosphorylate serine and threonine residues. PKC consists of six different isoforms, three of which are sensitive to intracellular calcium (α, β, and γ forms) and three that are not (δ, ε, and ζ forms). Neutrophils contain the α, β, and ζ forms but not the γ form. The calcium-dependent and DG-dependent PKC (PKC-β) responds to fMLP and phorbol ester stimulation by translocating from the cytosol to the membrane. It then phosphorylates a number of cytosolic proteins, such as those involved in the respiratory burst oxidase system. Additionally, PKC can specifically and transiently phosphorylate the myristolated alanine-rich C kinase substrate that may be important in regulating the attachment of actin filaments to the plasma membrane. fMLP can also activate the calcium-independent, DG-dependent and phosphatidyl serine-dependent PKC form but their function is unclear.

The MAP kinase (MAPK) reportedly is activated by the βγ subunits of the G-proteins by the activities of Ras and Raf. Recent literature suggests the involvement of high-intensity Ras signaling in inducing apoptosis (Bar-Sagi, et al, J. Mol. Cell Biol. 19(9):5892-901, 1999) as well as in promoting endothelial cell adherence. Raf is now posited with a central role in growth signals, including cell survival, growth and differentiation. This kinase pathway is also stimulated by C5a and IL-8 (Buhl et al., J. Biol. Chem. 270: 19828-19832, 1995; Knall et al., J. Biol. Chem. 271: 2832-2838, 1996). MAP kinase induces tyrosine phosphorylation of several regulatory proteins, such as the extracellular signal-regulated kinase (ERK)-1. Recent literature suggests that MAPK pathways are responsible for cytokine production; however, the activation of both TH-1 and TH-2 cytokines, as well as other pro-inflammatory molecules, such as C5a, IL-8 and FMLP, is dependent upon the trimeric G-protein signal transduction. Additionally, H-Ras and Faf-a, members of the MAPK pathway can act as negative regulators of integrin activity.

Phosphatidylinositol 3-linase (PI3K) is responsible for the formation of PI triphosphate (PIP₃) that is observed upon stimulation by FMLP. Elevated PIP₃ levels apparently contribute to the activation of the respiratory burst oxidase system and to actin polymerization in neutrophils, which is considered important in regulating cytoskeletal changes and cell migration. Recent literature (Rankin, et al, J. Exp. Med. 188(9):1621-32, 1998) has reported that elevated PI3 kinase levels also can promote degranulation of eosinophils, based upon G-protein signaling based activation of IL-5. Further, Sagi-Eisenberg, et al., Eur.J.Immunol, 1998.28: 3468-3478 suggest that G-protein signaling, using the intermediate pathways of PKC and PI3 kinases, may activate the FCεR receptor by IgE for the release of histamines and other pro-inflammatory cytokines involved in allergic airway hypersensitivity. Uckun, et al., J.of Biolog. Chemistry, Vol.274, No.38, September, 1999, pp.27028-27038 reports G-protein signaling in the JAK3 kinase pathway, through IgE/FCεR1 cross-linking, as leading to mast cell degranulation. Beaven, et al., J. of Immunology, 1998, 160: 5136-5144 report that G-protein signaling, through the activation of PKC and resulting uptake in Ca²⁺, also leads to secretion and degranulation of mast cells. Thus, G-protein may be essential for the down-stream activation of the FCεR1 upon IgE antigen challenge, and the corresponding ability to interfere with G-protein signaling, can be an important basis for down-stream inhibition of the activation of the FCεR receptor.

As mentioned above, integrins serve as a critical link connecting extracellular signals to intracellular pathways. Integrins have the capacity for bidirectional communication and can transmit signals from ‘outside-in’ and from ‘inside-out’. Inside-out signaling occurs when the cytoplasmic domains of the integrin receptor interact with intercellular proteins such as calreticulin, various serine/threonine kinases and small GTPase proteins such as R-Ras and RhoA. Inside-out signaling functions to increase the affinity of the integrin for its ligand. Inhibitors of the G protein and tyrosine kinase signal transduction pathways can prevent activating of the integrin to the high-affinity binding state.

The Focal adhesion kinase (FAK) is also involved in integrin mediated signal transduction. Upon interaction of integrins with the ECM, the tyrosine phosphorylation, and consequently the activity of FAK, is increased. Disruption of actin polymerization or RhoA function causes FAK activity to be downregulated.

### SUMMARY OF THE INVENTION

The present invention provides methods for treating a variety of indications involving integrin-mediated cell adhesion comprising contacting a cell containing an α6 integrin subunit with an VLA-6 integrin-mediated signal transduction pathway modification agent (“VLA6-IMSTPMA”) and forming a complex of agent with an α6 integrin subunit. Preferred agents are N-formyl-methionyl-leucyl (f-Met-Leu) peptides, which preferably are administered in a pharmaceutically acceptable carrier. The preferred f-Met-Leu peptides of the invention modulate VLA-6 integrin-mediated signal transduction. Particularly useful peptides are those having the formula f-Met-Leu-X where X is selected from the group consisting of Tyr, Tyr-Phe, Phe-Phe and Phe-Tyr, most preferably f-Met-Leu-Phe-Phe. Thus, preferred embodiments of the present invention provides a complex of an α6 integrin subunit with a peptide having the formula f-Met-Leu-X where X is selected from the group consisting of Tyr, Tyr-Phe, Phe-Phe and Phe-Tyr, most preferably f-Met-Leu-Phe-Phe.

In accord with the present invention, a method for treating an VLA-6 integrin-mediated pathological condition in a mammal comprises administering to the mammal an effective amount of VLA6-IMSTPMA, preferably a peptide having the formula f-Met-Leu-X where X is selected from the groups consisting of Tyr, Tyr-Phe, Phe-Phe and Phe-Tyr. As used herein, an “effective amount” means an amount that can modulate VLA-6 integrin-mediated signal transduction providing a therapeutic affect. As used herein, “modulate” means to affect the ability of a particular VLA-6 integrin to perform any of its functions including, for example, signaling, adhesion, fusion and internalization.

In accord with the present invention, the f-Met-Leu (“fML”) peptide forms a complex with an α6 subunit of VLA-6 integrin present on the surface of a cell. This complex blocks or modulates integrin function, preferably modulating the various downstream pathways used by integrins for outside-in or inside-out signal transduction.

The invention further provides a method to block or modulate the conventional pro-inflammatory response in a mammal, particularly downstream pro-inflammatory responses induced by pro-inflammatory agents such as, for example, C5a, fMLP, IL-4, IL6, IL-8, IL-10, IL-13 and TNFα or by the FCε receptor. The method comprises administering to a mammal an effective VLA-6 integrin-mediated signal transduction modulating amount of VLA6-IMSTPMA, preferably a peptide having the formula f-Met-Leu-X where X is selected from the group consisting of Tyr, Tyr-Phe, Phe-Phe and Phe-Tyr. The administration of the peptide can be before or after exposure to the pro-inflammatory agent.

In another embodiment of the present invention, a method for inhibiting cancer cell metastasis. The method comprises contacting a cell with an effective VLA-6 integrin-mediated signal transduction modulating amount of VLA6-IMSTPMA, preferably a peptide having the formula f-Met-Leu-X where X is selected from the groups consisting of Tyr, Tyr-Phe, Phe-Phe and Phe-Tyr. Preferably, a method for inhibiting cancer cell metastasis in a mammal comprises administering to the mammal an effective metastasis inhibiting amount of a peptide having the formula f-Met-Leu-X where X is selected from the groups consisting of Tyr, Tyr-Phe, Phe-Phe and Phe-Tyr. Although not being bound by any theory, it is thought that the fML peptide inhibits the ability of the cancer cell to attach and invade at another tissue site.

In another embodiment of the present invention, a method for treating coronary heart disease in a mammal is provided. The method comprises administering to the mammal an effective VLA-6 integrin-mediated signal transduction modulating amount of VLA6-IMSTPMA, preferably a peptide having the formula f-Met-Leu-X where X is selected from the groups consisting of Tyr, Tyr-Phe, Phe-Phe and Phe-Tyr. Although not being bound by any theory, it is thought that the fML peptide prevents platelet spreading and aggregation and, thus, also is useful in the treatment of diseases such as thrombosis, atherosclerosis, and the like.

In certain preferred embodiments of the present invention, patients can benefit by administering the VLA6-IMSTPMA of the present invention in combination with a second active ingredient. Particularly useful other active ingredients for such combination in accord with the present invention are for example, antileukotrienes, beta2 agonists, corticosteroids, chemotherapuetics, etc. Preferably, the peptide and any other ingredient are administered in a pharmacologically acceptable carrier, which is sterile and non-pyrogenic.

### BRIEF DESCRIPTION OF DRAWINGS

FIG. 1A-FIG. 1B are graphs showing the relationship between the DNA content of normal human peripheral blood mononuclear cells and the amount of fluoresceinated HK-X (f-Met-Leu-Phe-Phe) binding to the surface of the cells. FIG. 1A shows lymphocytes stimulated with 6 μg Concanavalin A (ConA) at 24 hours after addition of 100 nM FITC-labeled HK-X to the cell culture; and FIG. 1B shows lymphocytes stimulated with 6 μg ConA at 120 hours after addition of 100 nM FITC-labeled HK-X to the cell culture.

FIG. 2 is a graph showing the binding of FITC-labeled HK-X to human peripheral blood nucleated cells. The level of HK-X binding to peripheral mononuclear blood cells (PMNs)/Basophils (Baso) is represented by asterisks and the level of HK-X binding to Eosinophils is represented by dots.

FIG. 3 is a graph showing the binding of FITC-labeled HK-X to rat peritoneal mast cells. The level of HK-X binding to two separate preparations of rat peritoneal rat cells are represented by squares (mast cell preparation number 1) and triangles (mast cell preparation number 2) and the level of HK-X binding to PMNs is represented by open circles.

FIG. 4 is a representation of an autorad of a polyacrylamide gel showing the 165 kDa protein (³⁵S-methionine labeled) that was purified using HK-X substituted sepharose.

FIG. 5A-FIG. 5B show spectrum obtained from MALDI analysis of the 160 kDa protein isolated from a gel similar to that illustrated in FIG. 4.

FIG. 6 is a representation of a Western Blot showing that antibodies specific for the integrin subunits α6 and β1 recognize proteins purified using HK-X substituted sepharose.

FIG. 7 is an outline of the methodology used to obtain information regarding the level of protein kinases present after stimulation of cells with HK-X alone or HK-X in combination with a cytokine or pro-inflammatory agent.

### DETAILED DESCRIPTION OF THE INVENTION

In accord with the present invention, VLA-6 integrin-mediated signal transduction pathway modification agents (“VLA6-IMSTPMA agents”) have been found to have surprising activity for modulating integrin function, particularly VLA-6 integrin-mediated signal transduction. As a result, such agents are useful for treatment of a variety of indications resulting from VLA-6 integrin-mediated responses. Examples of such indications include asthma, inflammation, psoriasis, rheumatoid arthritis, inflammatory bowel disease, coronary heart disease, thrombosis, atherosclerosis, ARDS, gout, tumor antigenesis, meconium aspiration and anterior uveitis. Prefered VLA6-IMSTPMA agents are certain small peptides having the formula f-Met-Leu-X where X is selected from the group consisting of Tyr, Tyr-Phe, Phe-Phe and Phe-Tyr

Preferred VLA6-IMSTPMA agents, in accord with the present invention, can disrupt certain pro-inflammatory responses of human peripheral blood cells that have been stimulated by pro-inflammatory agents or molecules.

Preferred VLA6-IMSTPMA agents of the present invention can bind to α6 integrin subunits or receptors on the surface of a cell involved in various disease states. Such disease states include those diseases or conditions resulting from chronic or inappropriate inflammation such as asthma, organ rejection, and the like; diseases involving platelet aggregation or spreading such as coronary heart disease, thrombosis, atherosclerosis and the like; and diseases involving metastasis of cells such as cancer.

A preferred embodiment of the present invention provides a cell surface complex between an α6 integrin receptor and a VLA6-IMSTPMA agent. Particularly preferred are complexes of fML peptides with the VLA-6 integrin receptor. Most preferred are peptides which specifically bind and modulate VLA-6 mediated signal transduction without affecting the signal transduction mediated by other integrin receptors.

Cells involved in inflammatory conditions include pro-inflammatory mediating cells such as lymphocytes, particularly activated T-cells, granulocytes such as eosinophils, basophils, neutrophils, and fixed tissue cells such as mast cells and the like.

Cells involved in coronary heart diseases include for example, endothelial cells, smooth muscle cells, platelets, monocytes, leukocytes, etc.

Cells involved in cancer metastasis can include for example, cells of the breast, prostate, ovary, central nervous system, brain, colon, lung, skin, etc.

As used herein, pro-inflammatory responses include secretion or degranulation of pro-inflammatory mediating cells and release of leukotrienes, histamines, and other cytokines. Such responses also include infiltration of eosinophils, basophils and mast cells into inflammatory tissues as a result of chemotactic adhesion, migration and aggregation of lymphocytes, eosinophils, basophils, mast cells and neutrophils. Vascular permeability at the site of inflammation and increased production of IgE, IgG and IgA, and their respective FC receptors, also can be associated with pro-inflammatory responses.

Inhibition of pro-inflammatory responses can thus include decrease of degranulation and release of leukotrienes, histamines and other cytokines by pro-inflammatory mediating cells, or complete cessation in preferred embodiments, following peptide-integrin binding according to the present invention. Infiltration and migration of pro-inflammatory mediating cells can also be greatly reduced, or completely inhibited. Vascular permeability at the site of inflammation and IgE levels also can be reduced.

VLA-6 plays an important role in regulating adhesion and migration of monocytes, eosinophils, B cells and activated T lymphocytes to sites of chronic inflammation.

The fML peptides of the present invention, particularly fMLPP, preferably bind simultaneously to both the FPR and the alpha 6 subunit of VLA-6 to provide inhibition of inflammatory mediators under conditions of challenge by pro-inflammatory agents. Although not being bound by theory, this is thought due to binding to VLA-6 and the interrelationships in the signal pathways between the integrins, pro-inflammatory molecules and the FPR. Further, potential cross-talk involvement of CD18, 20, 40, 41 and 61 has far-ranging implications in the treatment of a host of major disease indications. Moreover, the involvement with the FPR through G-protein signaling has strongly synergistic implications, given the numerous cross-communication links between integrin signaling with the activation of the FPR in relation to fMLP, C5a, IL-8 and other pro-inflammatory molecules.

VLA-6 (very late antigen-6) is known to be found on the following cells: fibroblasts, endothelial cells, epithelial cells, platelets, T lymphocytes and neutrophils.

The preferred fML peptides antagonize the FPR on cells stimulated by pro-inflammatory agents, and simultaneously bind to the alpha 6 subunit of VLA-6. This action can initiate a number of significant changes in both the FPR and chemokine/cytokine directed Ras-Raf-MAPK-ERK-JUNK kinase pathway as well as the integrin receptor directed FAK-Ras-Raf-MEK pathway. These pathways are not mutually exclusive, and enjoin cross-talk at various points in their changed signals and calcium flux. These effects can be synergistic. Direct binding of the preferred fML peptides to the integrin can negatively effect its capacity to form focal adhesion complexes, become activated, and send downstream kinase signals.

A strong absence of binding and migration of pro-inflammatory cells to the site of inflammation (including reduction in the binding of ECMs) is consistent with in vivo evidence of integrin receptor antagonism affected by preferred fML peptides. Data suggests that preferred fML peptides also down-regulate the pro-inflammatory response to inflammatory agents that remain resident in the tissue surrounding and at the site of inflammation through antagonism of the FPR, as evidenced in-vivo by the strong clearing of cellular infiltrate, reduction of mucus plugs and reduction of ICAM and VCAM.

Preferred compounds of the present invention exhibit no toxicity to vital organs such as heart, liver, lungs, kidneys, brain and gut.

The peptides of this invention can be prepared by conventional small peptide chemistry techniques. The peptides when used for administration are prepared under aseptic conditions with a pharmaceutically acceptable carrier or diluent.

The pharmaceutical compositions may conveniently be presented in unit dosage form and prepared for each type of indication resulting from integrin-mediated responses that is to be treated. The compositions may be prepared by any of the methods well known in the art of pharmacy. Methods typically include the step of bringing the active ingredients of the invention into association with a carrier that constitutes one or more accessory ingredients.

For example, doses of the pharmaceutical compositions will vary depending upon the subject, type of indication to be treated, and upon the particular route of administration used. Dosages of active peptide when treating acute integrin-mediated responses can range from 0.1 to 100,000 μg/kg a day, more preferably 1 to 10,000 μg/kg. Most preferred dosages range from about 1 to 100 μg/kg of body weight, more preferably from about 1 to 20 μg/kg and most preferably 10 to 20 μg/kg. Dosages of active peptide when treating chronic integrin-mediated responses can range from 0.1 to 100,000 μg/kg a day, more preferably 1 to 10,000 μg/kg. Most preferred dosages range from about 1 to 1000 μg/kg of body weight, more preferably from about 1 to 100 μg/kg and most preferably 50-70 μg/kg. Doses are typically administered from once a day to every 4-6 hours depending on the severity of the condition. For acute conditions, it is preferred to administer the peptide every 4-6 hours. For maintenance, it may be preferred to administer only once or twice a day. Preferably, from about 0.18 to about 16 mg of peptide are administered per day, depending upon the route of administration and the severity of the condition. Desired time intervals for delivery of multiple doses of a particular composition can be determined by one of ordinary skill in the art employing no more than routine experimentation.

Routes of administration include oral, parenteral, rectal, intravaginal, topical, nasal, ophthalmic, direct injection, etc. In a preferred embodiment, the peptides of this invention are administered to the patient in an integrin inhibiting effective amount. An exemplary pharmaceutical composition is an effective amount of a peptide in accord with the present invention that causes a modulation of integrin-mediated signal transduction, typically included in a pharmaceutically acceptable carrier.

The term “pharmaceutically acceptable carrier” as used herein, and described more fully below, includes one or more compatible solid or liquid filler diluents or encapsulating substances that are suitable for administration to a human or other animal. In the present invention, the term “carrier” thus denotes an organic or inorganic ingredient, natural or synthetic, with which the molecules of the invention are combined to facilitate application. The term “effective amount” is that amount of the present pharmaceutical compositions which produces an effect on the particular condition being treated by modulating integrin-mediated signal transduction. Various concentrations may be used in preparing compositions incorporating the same ingredient to provide for variations in the age of the patient to be treated, the severity of the condition, the duration of the treatment and the mode of administration.

The carrier must also be compatible. The term “compatible”, as used herein, means that the components of the pharmaceutical compositions are capable of being commingled with a small peptides of the present invention, and with each other, in a manner such that does not substantially impair the desired pharmaceutical efficacy.

The small peptides of the invention are typically administered per se (neat). However, they may be administered in the form of a pharmaceutically acceptable salt. Such pharmaceutically acceptable salts include, but are not limited to, those prepared from the following acids: hydrochloric, hydrobromic, sulfuric, nitric, phosphoric, maleic, acetic, salicylic, p-toluene-sulfonic, tartaric, citric, methanesulphonic, formic, malonic, succinic, naphthalene-2-sulfonic, and benzenesulphonic. Also, pharmaceutically acceptable salts can be prepared as alkaline metal or alkaline earth salts, such as sodium, potassium or calcium salts of the carboxylic acid group. Thus, the present invention provides pharmaceutical compositions, for medical use, which comprise peptides of the invention together with one or more pharmaceutically acceptable carriers thereof and optionally any other therapeutic ingredients.

The compositions include those suitable for oral, rectal, intravaginal, topical, nasal, ophthalmic or parenteral administration, all of which may be used as routes of administration using the materials of the present invention. Pharmaceutical compositions containing peptides of the present invention may also contain one or more pharmaceutically acceptable carriers, which may include excipients such as stabilizers (to promote long term storage), emulsifiers, binding agents, thickening agents, salts, preservatives, solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the peptide of this invention, its use in pharmaceutical preparations is contemplated herein. Supplementary active ingredients can also be incorporated into the compositions of the present invention.

Compositions suitable for oral administration are typically prepared as an inhalation aerosol, nebule, syrup or tablet. Compositions suitable for topical administration typically are prepared as a cream, an ointment, or a solution. For treating an acute integrin-mediated response, the concentrations of the peptide active ingredient in such compositions is typically less than 1000 μg/ml, more preferable less than 500 μg/ml, and most preferably from about 200 to 400 μg/ml. For treating a chronic integrin mediated response, the concentrations of the peptide active ingredient in such compositions is typically less than 3 mg/ml, more preferable less than 2 mg/ml, and most preferably from about 1 to 1.5 mg/ml.

Compositions of the present invention suitable for inhalation administration may be presented, for example, as aerosols or inhalation solutions. An example of a typical aerosol composition for treating acute integrin-mediated responses consists of about 0.1 to 100 μg of microcrystalline peptide suspended in a mixture of trichloro-monofluoromethane and dichlorodifluoromethane plus oleic acid, per dose. A more preferable amount of microcrystalline peptide in the composition is 1 to 50 μg, and most preferable is 10 to 20 μg per dose of the aerosol composition. An example of a typical aerosol composition for treating chronic integrin-mediated responses consists of about 0.1 to 1000 μg of microcrystalline peptide suspended in a mixture of trichloro-monofluoromethane and dichlorodifluoromethane plus oleic acid, per dose. A more preferable amount of microcrystalline peptide in the composition is 1 to 100 μg, and most preferable is 50 to 70 μg per dose of the aerosol composition. An example of a typical solution consists of the desired quantity of peptide dissolved or suspended in sterile saline (optionally about 5% v/v dimethylsulfoxide (“DMSO”) for solubility), benzalkonium chloride, and sulfuric acid (to adjust pH).

Compositions of the present invention suitable for oral administration also may be presented as discrete units such as capsules, cachets, tablets or lozenges, each containing a predetermined amount of the peptide of the invention depending on the type of integrin mediated response to be treated, or which may be contained in liposomes or as a suspension in an aqueous liquor or non-aqueous liquid such as a syrup, an elixir, or an emulsion. An example of a tablet formulation base includes corn starch, lactose and magnesium stearate as inactive ingredients. An example of a syrup formulation base includes citric acid, coloring dye, flavoring agent, hydroxypropylmethylcellulose, saccharin, sodium benzoate, sodium citrate and purified water.

Compositions suitable for parenteral administration conveniently comprise a sterile aqueous preparation of the molecule of the invention, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In aqueous solutions, up to about 10% v/v DMSO or Trappsol can be used to maintain solubility of some peptides. Also, sterile, fixed oils may be conventionally employed as a solvent or suspending medium. For this purpose, a number of fixed oils can be employed including synthetic mono- or diglycerides. In addition, fatty acids (such as oleic acid or neutral fatty acids) can be used in the preparation of injectibles. Further, Pluronic block copolymers can be formulated with lipids at 4° C. for compound injection on a time release basis from solid form at 37° C. over a period of weeks or months.

Compositions suitable for topical administration may be presented as a solution of the peptide in Trappsol or DMSO, or in a cream, ointment, or lotion. Typically, about 0.1 to about 2.5% active ingredient is incorporated into the base or carrier. An example of a cream formulation base includes purified water, petrolatum, benzyl alcohol, stearyl alcohol, propylene glycol, isopropyl myristate, polyoxyl 40 stearate, carbomer 934, sodium lauryl sulfate, acetate disodium, sodium hydroxide, and optionally DMSO. An example of an ointment formulation base includes white petrolatum and optionally mineral oil, sorbitan sesquioleate, and DMSO. An example of a lotion formulation base includes carbomer 940, propylene glycol, polysorbate 40, propylene glycol stearate, cholesterol and related sterols, isopropyl myristate, sorbitan palmitate, acetyl alcohol, triethanolamine, ascorbic acid, simethicone, and purified water.

In order that the invention described herein may be more fully understood, the following examples are set forth. It should be understood that these examples are for illustrative purposes only and are not to be construed as limiting this invention in any manner.

### DETAILED MATERIALS AND METHODS FOR EXAMPLES 1-3

1. Isolation of Cells

Rat peritoneal mast cells were isolated by infusion of 35 ml of Tyrode's Solution into the peritoneal cavity of anesthetized rats. The rats were then sacrificed by injection of an overdose of anesthetic. The peritoneal cells were harvested, placed into 15 ml centrifuge tubes. The cells were pelleted by centrifugation at 250×g for 10 min at room temperature.

Human peripheral blood mononuclear and polymorphonuclear cells were isolated from peripheral blood obtained from normal donors. The blood was collected in heparin. The various cell types were isolated by centrifugation over Ficoll-Hypaque at 500×g for 60 min at room temperature. Each fraction was harvested, pooled separately and washed 1× in RPMI 1640 with antibiotics.

2. Metabolic Labeling of Cells with ³⁵S-Methionine

Cells were adjusted to 1×10⁷ per ml of methionine-less RPMI 1640 media containing 10 μCi of ³⁵S-methionine and held overnight at 37° C. in the presence of 5% CO₂.

3. Harvesting of Cells and Preparation of Crude Membranes

Cells were washed 3× in PBS and subsequently lysed by sonication in Hepes buffer, pH 7.2 containing 0.3% NP40 and proteinase inhibitor cocktail. The resulting cellular preparation was centrifuged at 600×g for 10 min and the supernatant collected for further analyses.

4. Sepharose® and Sepharose® HK-X Chromatographic Separation of Various Cellular Proteins

The cellular preparation was passed through a column of Sepharose® unsubstituted resin or to Sepharose® HK-X resin and divided into two portions, A and B.

Aliquot A: was passed over HK-X substituted Sepharose® column. Columns washed and then eluted with buffer containing HK-X (5 mg/ml), and then with 0.1 M glycine, pH 2.5.

Aliquot B was bound to an HK-X substituted Sepharose® column in the presence of soluble HK-X and the proteins eluted. Each fraction was concentrated and Lyophilized.

The approach undertaken in this step involved binding of HK-X to a Sepharose® resin to make an HK-X substituted resin. Prior to exposure to HK-X substituted resin, the labeled cellular protein mixture was passed over a resin not substituted with HK-X to remove any protein species reacting with the native resin. Thus, when the cellular proteins including the receptor proteins were passed through the HK-X substituted resin under proper ionic environments, the receptor proteins (for the HK-X receptor) among the other proteins bound tightly with the HK-X. The resin was washed with a gentle agent, such as phosphate buffer at neutral pH, to remove any low affinity binding proteins. Subsequently, the resin was exposed to an excess amount of free HK-X to competitively elute receptor proteins bound to the resin. The radioactive proteins released at each of these steps was concentrated and analyzed on a 12% SDS-PAGE system as detailed in the following step.

5. 12% SDS-PAGE

25 μL of the radioactive cellular preparation containing 250 cpm to 2000 cpm radioactivity was applied to each lane of the gel. The gel was run at 90 V at 30 mA until good resolution of colored standards was obtained. The standards were phosphorlyase b (MW=94,000); bovine serum albumin (MW=68,000); ovalbumin (MW=43,000); carbonic anhydrase (MW=30,000); and soybean trypsin inhibitor (MW=21,000).

6. Estimation of Molecular Weight of Resolved Radioactive Proteins

The relative mobilities were calculated for the standards and for each distinct molecular weight species visualized on the gel. A plot of log molecular weight of the standards was plotted against relative mobility for each standard. The data were entered in PRISM software and the molecular weights of the unknown proteins were predicted from a Standard Curve Program. The results were compared to FPR receptor proteins published in the literature (Goetzl et al., Biochemistry 20:5717-5722, 1981).

### Example 1

Binding of Labeled HK-X to Mitogen Activated Human Peripheral Blood Mononuclear Cells

Peripheral blood lymphocytes were stimulated with the mitogen Concanavalin A (ConA) at 24 hours or at 120 hours after being placed in culture. The cells were then either exposed to the 100 nM FITC-labeled f-Met-Leu-Phe-Phe (HK-X) or were exposed to a control (vehicle not containing peptide). Cells were also stained with DAPI for cell cycle determination. Cells were then analyzed by flow cytometry.

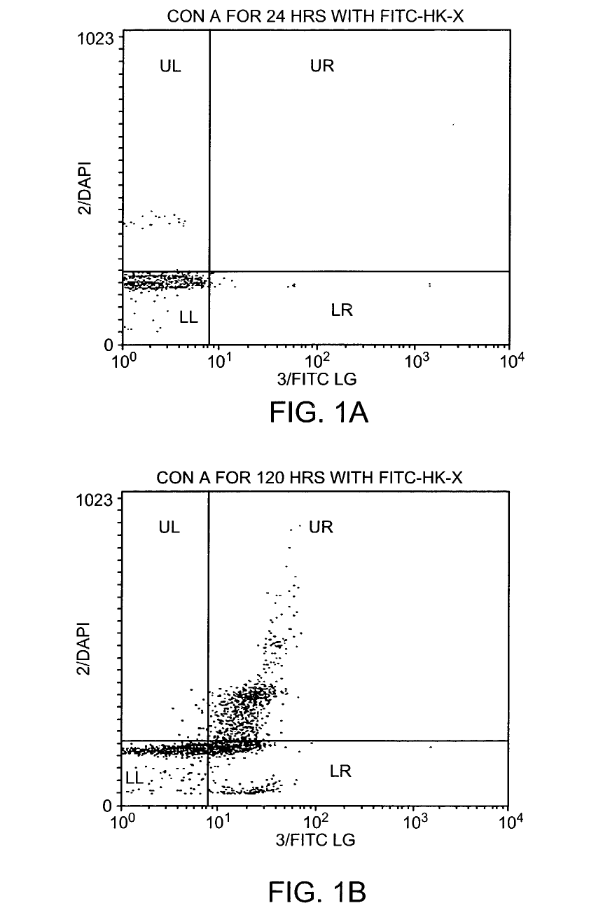

FIGS. 1A-1B show the relationship between activated lymphocytes by ConA and the appearance of binding sites for FITC-labeled HK-X. The four quadrants reveal the following characteristics:

| | |
|---|---|
| a.) | the upper left quadrants denote cells with greater than 1n content of |
| | DNA and increased levels of FITC HK-X binding above background |
| | levels; |
| b.) | the upper right quadrants denote cells that contain greater than 1n |
| | DNA content and have FITC-ligand binding greater than background; |
| c.) | the lower right quandrants contain cells that have 1n DNA content |
| | but have bound FITC-ligand above background levels; and |
| d.) | the lower left quadrants contain cells with 1n DNA content and |
| | background levels of FITC-ligand. |

As is apparent from examination of FIGS. 1A and 1B, exposure to the plant lectin or mitogen stimulates cells to enter into the cell cycle and express binding sites to the FITC-labeled HK-X. The longer culture period of 120 hours (FIG. 1B) allowed a greater portion of the cells to enter the cell cycle (compared to 24 hours in FIG. 1A). The most accurate determination of background levels of endogenous fluorescence was obtained by setting the thresholds (quadrants) using cells cultured with ConA but not stained with the FITC-labeled HK-X.

Combining analyses of the cell surface concomitantly with DNA content of cells it can be determined that activated cells residing in either the G0/G1 (50% of the cells) or in the S+G2+M (100% of the cells) phases of the cell cycle demonstrated HK-X binding. (See FIG. 1B) Lymphocytes cultured with control materials, vehicle or culture media alone did not develop receptors for HK-X (See FIG. 1A). Thus, entry into the cell cycle as determined by initiation of DNA synthesis lead to an increase in the amount of binding of HK-X to the cell surface.

The effects of addition of HK-X (as compared to a control of vehicle without peptide) to cultures of normal human peripheral blood mononuclear cells with and without the plant lectin, Con A, were also evaluated. It was found that HK-X did not alter the fraction of cells entering the cell cycle and did not effect cell viability. However, HK-X induced apoptosis in a significant population of cells (˜30%) stimulated with Con A as determined by the presence of a sub G0/G1 population in the DNA profile. In cultures where the cells were not stimulated to enter the cell cycle (without Con A), the levels of sub G0/G1 were similar in HK-X treated and control cultures (<7%).

### Example 2

Binding of Labeled HK-X to Other Cell Types

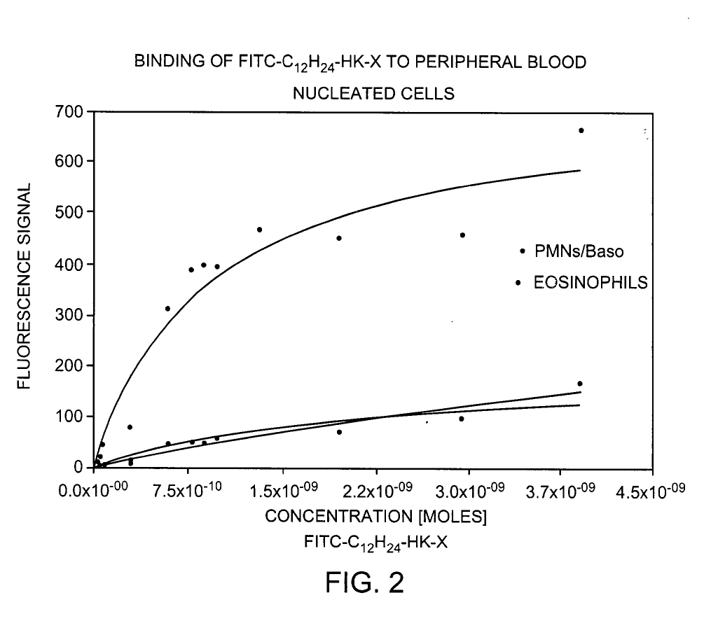

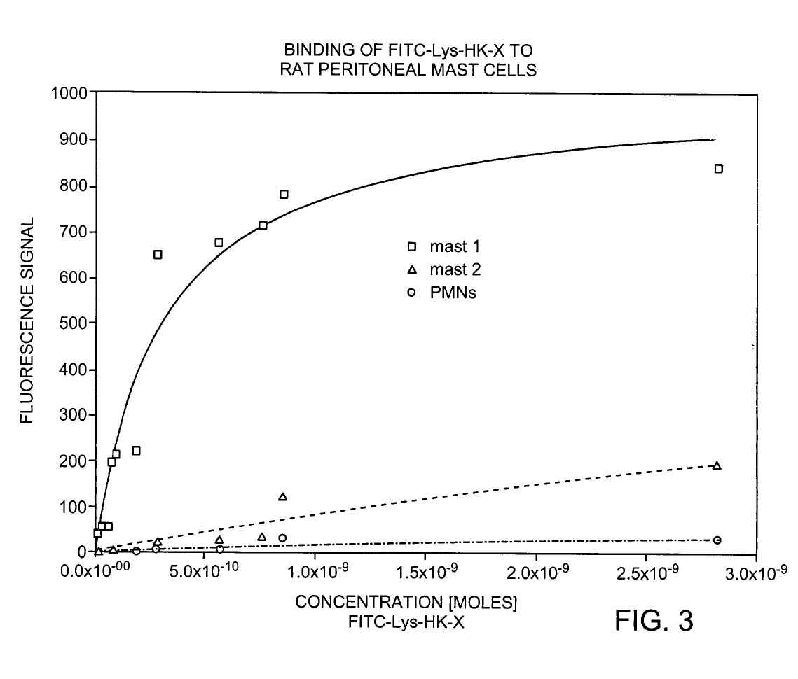

Other cell types were also tested for the ability to bind FITC labeled HK-X on their cell surface. Human peripheral blood basophils, neutrophils, and eosinophils had large numbers of HK-X receptors compared to freshly isolated monocytes and lymphocytes (FIG. 2). Since normal mast cells are difficult to isolate from humans, at peritoneal mast cells were used to determine whether or not mast cells could bind FITC-HK-X. Indeed, freshly isolated mast cells bound numbers of FITC-HK-X roughly equivalent to that of human eosinophils (FIG. 3).

### Example 3

Identification and Characterization of HK-X Binding Receptors on Cell Surfaces

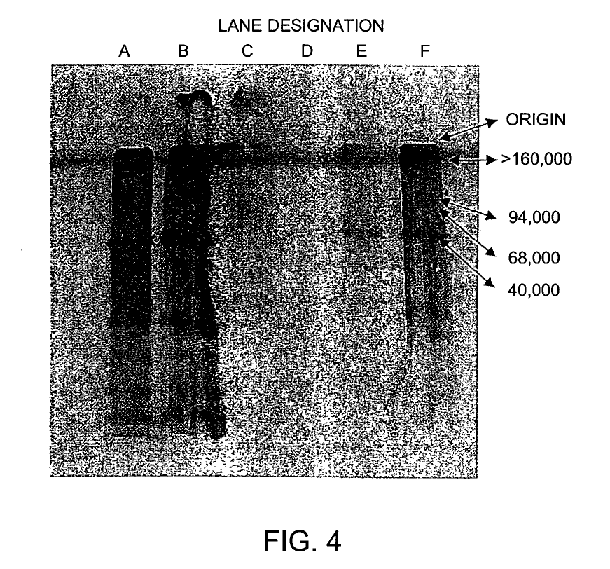

An affinity purification procedure was used to identify the cell surface proteins with HK-X binding activity. Cells were harvested from peripheral blood, washed, and placed into cell culture media without exogenous methionine. ³⁵S-methionine was added to the cells in order to label newly synthesized proteins, which were passed over HK-X substituted Sepharose®. Proteins bound to the HK-X Sepharose® can be specifically recovered by competition with free HK-X or with mild acid (pH 2.5) treatment. The radiolabeled proteins recovered from the affinity column were analyzed by SDS-PAGE, which allowed the determination of the molecular weight of each protein species with HK-X binding activity. FIG. 4 demonstrates the result of a representative experiment. In lane F, 4 major proteins were recovered under pH 2.5 elution conditions from the affinity column. The distribution of molecular weights of 40, 68 and 94 Kd is consistent with the fact that these subunits belong to the formyl peptide receptor (FPR) family.

FIG. 4 shows the result of a representative experiment. All proteins present in the cell lysate are shown in Lane A. In Lane B, the unbound material from the Sepharose® column without HK-X substitution shows a pattern of protein band distribution similar to the entire cell lysate. Lane C contains the pre-elution material. Lane D is a blank lane. Lane E, shows the protein bands obtained when the column was eluted in the presence of 1 mg of HK-X (competitor). Lane F, shows the four bands obtained when the column was eluted using pH 2.5. The molecular weights are estimated to be ˜165,000, ˜94,000, ˜68,000, ˜40,000 Daltons, respectively. This experimental condition established the specificity of the binding. The 94, 68 and 40 Kd bands are subunits of the FPR receptor. The 165 Kd band was eluted from the HK-X column using pH 2.5 acid conditions.

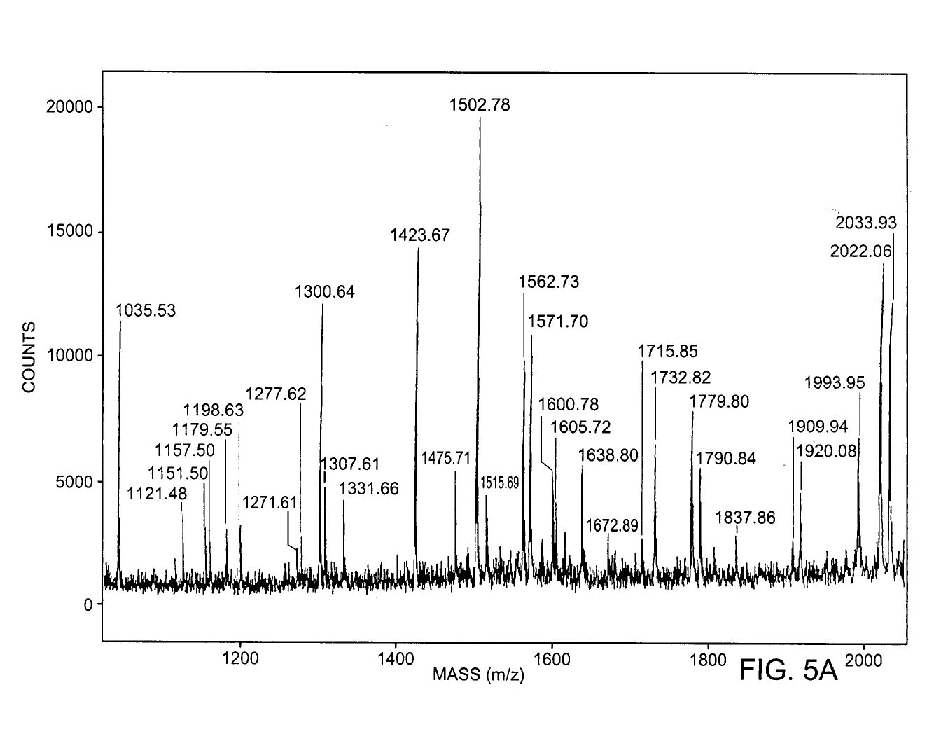

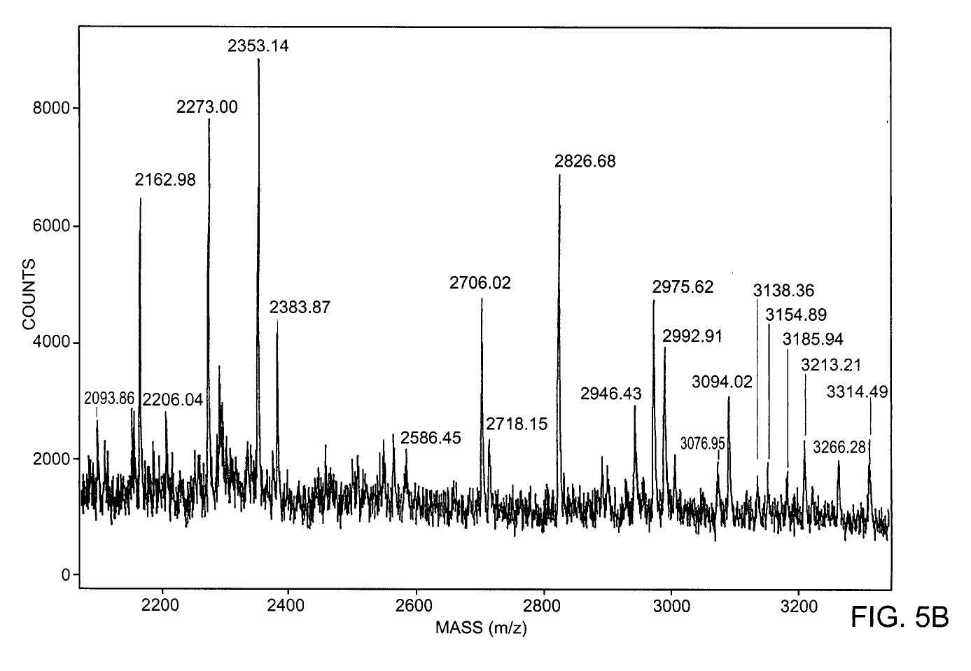

The 165 Kd species required additional analyses in order to obtain its identity. To that end, MALDI analysis was performed at a commercial analytical laboratory on a 165 Kd species isolated from the affinity column with acid treatment. Fifty-two peptides from the 165 Kd species were analyzed with 31% of putative protein sequences covered (FIG. 5A-5B). The amino acid sequence of the peptides was consistent with that of an integrin of the alpha family (Hiraiwa et al., Blood 69: 560-564, 1987). A ProFound database search was performed and statistically, the best sequence match suggested that the alpha chain belonged to the alpha 2b-platelet glycoprotein or a member of a related VLA integrin family. FIG. 5A shows the mass/charge values for peptides in the 1-2 Kd range. FIG. 5B shows the mass/charge values for peptides in the 2-3.5 Kd range. Interestingly, integrins are heterodimeric proteins where an alpha subunit is combined with a beta subunit on the cell surface in order to have a fully functional integrin. Under the conditions of the experiment no beta subunit was retained on the affinity resin. Thus, it would appear that HK-X binding is performed by the alpha subunit, not the beta subunit. However, not finding a beta chain associated with the alpha subunit was not due to a lack of sensitivity of the methodology. Therefore, the explanation for the presence of only an alpha subunit of an integrin synthesized by leukocytes necessitated further experimentation.

### Example 4

Identification of HK-X Binding Receptors on Cell Surfaces; Western Blot Analyses of Constitutively Expressed Integrins

Human peripheral blood cells were purified by buoyant density centrifugation on a Ficoll-Hypaque cushion after being separated from platelet rich plasma. Both the platelets and the peripheral blood leukocytes were washed and then lysed with 0.1% NP-40 in the presence of a commercially available cocktail of protease inhibitors (ICN, Irvine, Calif.). Lysates were exposed to HK-X substituted Sepharose® resins and elution performed with free HK-X (competitive elution, defines specificity of binding) or with pH 2.5 (acid elution, high stringency conditions). The eluates were dialyzed, concentrated and lyophilized. The reconstituted eluates were subjected to SDS-PAGE followed by transfer of the separated proteins onto nylon membranes for Western Blot analysis. Subunits of the VLA integrins were detected with antibodies directed to the α1, α2, α3, α4, α5, α6 as well as β1, β2 and β3 subunits. The specific antibodies used for this experiment are shown below in Table 1.

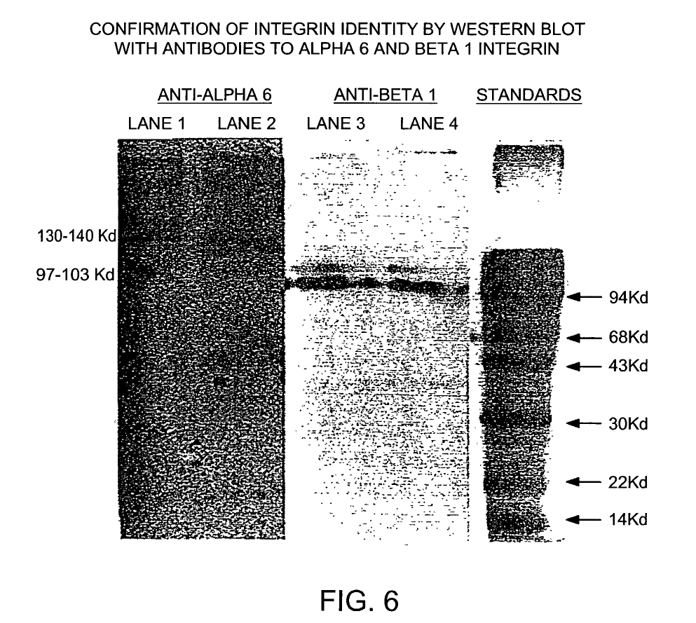

The results of the Western Blot experiment can be seen in FIG. 6. The α6 subunit was the only a subunit that was observed in both leukocytes and platelets recovered under competitive and acid conditions. The β1 subunits were observed in the same preparations as the β6 subunits. In FIG. 6, lanes 1 and 2 show platelet and leukocyte preparations probed with an antibody specific for the α6 integrin subunit, lanes 3 and 4 show platelet and leukocyte preparations probed with an antibody specific for the β1 integrin subunit and the far right lane shows the molecular weight protein standards. Binding of the primary antibodies to the integrin subunits was detected using rabbit anti-goat secondary antibody labeled with HRP (horse radish peroxidase). The protein molecular weight standards were blotted to the nylon membrane and then stained with coomassie blue.

TABLE 1
Specificity of Integrin Antibodies used for Western Blot analysis.

| Company | Class of Ab | Host | Specificity | Made Against |
|---|---|---|---|---|
| Santa Cruz | IgG | goat | integrin α1 | amino Terminus |
| Santa Cruz | IgG | goat | integrin α2 | amino Terminus |
| Santa Cruz | IgG | goat | integrin αIIb | carboxy Terminus |
| Santa Cruz | IgG | goat | integrin α3 | amino Terminus |
| Santa Cruz | IgG | goat | integrin α4 | carboxy Terminus |
| Santa Cruz | IgG | goat | integrin α5 | carboxy Terminus |
| Santa Cruz | IgG | goat | integrin α6 | amino Terminus |
| Santa Cruz | IgG | goat | integrin β1 | full length |
| Santa Cruz | IgG1 | mouse | integrin β3 | full length |

Sant Cruz Biotechnology Inc., 2161 Delaware Avenue, Santa Cruz, CA

The distribution of VLA-6 (very late antigen-6) on the surface of various cell types is shown in Table 2.

TABLE 2
Cells Types Bearing VLA-6: Distribution and Amount

| | RELATIVE | |
|---|---|---|
| BLOOD AND TISSUE | AMOUNT PER | PERCENT OF β1 |
| CELLS | CELL@ | INTEGRIN** |
| PLATELETS | 46^{a} | 33 |
| B CELLS | 1 | 2 |
| T CELLS | 55 | 34 |
| THYMOCYTES | 13 | 25 |
| MONOCYTES | 390 | 51 |
| GRANULOCYTES | ? | ? |
| EPIDERMAL CELLS | +^{b} | + |
| ENDOTHELIAL CELLS | + | + |
| CULTURED T CELLS (3 | 11^{c} | 13 |
| DAYS) | | |
| CULTURED T CELLS (4 | 13 | 5 |
| WEEKS) | | |

@ Denotes the log mean fluorescent intensity of the VLA-6 per cell obtained from flow cytometric analysis.
**Denotes the fraction of all β1 molecules bound to α6 subunits.
+ Denotes that VLA-6 is present but fluorescent intensity and distribution is not available.
^{a}Hemler, M., Ann. Rev. Immunol., 8: 365-400, 1990.
^{b}Staquet, M. R., et al., Exp. Cell Res., 187: 277-283, 1990.
^{c}Hemler, M., et al., Eur. J. Immunol., 15: 502-508, 1985.

### Example 5

Effects of HK-X Administration to Normal Mice

When HK-X was administered to mice at concentrations ranging from 10 μg to 1000 μg per adult mouse, no alterations were observed in the distribution or numbers of nucleated cells of the peripheral blood. Secondly, the IgM and IgG antibody secreting cell responses in mice immunized with sheep red cells and treated with HK-X prior to and after immunization show no alteration in the numbers of antibody forming cells (PFC). In order to have increased numbers of IgM PFC, B cell growth factors leading to proliferation and differentiation of the specific B cells are required. In addition, the IgG PFC response requires the production of IL-1 by monocytes, processing and presentation of antigen by accessory cells, secretion of IL-2 by T cells, and secretion of cytokines leading to gene rearrangement in the responding B cells and generation of long-lived antigen specific T and B cells.

HK-X did not down regulate production of required cytokines. Furthermore, HK-X did not interfere with cellular cooperation, which depends heavily on physical association between responding cells. The specificity of association among cells depends on binding of integrins on the interacting cell surfaces to collagens, laminins and fibronectins within the interstitial tissues to dictate proper 3D orientation of the cells one to another.

HK-X did not promote or cause other disruptive signals interfering with the interaction of T and B cells. T and B cells within central immune (thymus) or peripheral immune tissues (spleen) and one set of receptors for B cells (surface immunoglobulin) were not negatively effected. Further, the data show that HK-X did not negatively affect the proliferation and differentiation of hematopoietic precursors of neutrophils, basophils, monocytes and lymphocytes.

The process involved in production of nucleated cells in the bone marrow and their release to the peripheral blood depends heavily on the actions of GM-CSF, G-CSF and M-CSF, among other cytolanes. Because the numbers of each of these cell types were well within normal ranges, the synthesis and secretion of these hematopoietic cytokines were not compromised by the administration of HK-X. Similar observations were made for the central lymphoid tissue (thymus) and the secondary tissue (spleen). Liver and kidney function was unaffected by HK-X, even though hepatocytes have FPR for HK-X.

### Example 6

Therapeutic Efficacy of HK-X Administration to Acute and Chronic Asthmatic Mice

In vivo mouse models of asthma have been established which mimic key morphologic and physiologic features of human disease (Henderson et al., J. Exp. Med., 184:1483-1494, 1996). The availability of specific therapeutic reagents enable further experimental manipulation of the mouse model in a systematic manner before, during and after induction of pulmonary inflammation.

In pathological situations HK-X has a significant inhibitory effect on mast cell degranulation both in vivo and in vitro. Eosinophil numbers were dramatically reduced in the lung during treatment with HK-X. In the mouse model of acute asthma described above, HK-X was administered intranasally for only three days during concomitant allergen challenge. Down-regulation or inhibition of mucus cell differentiation and subsequent mucus production in the mouse lung undergoing asthmatic pathological challenge was observed.

In the acute asthma model, HK-X interrupted the emigration of inflammatory cells into the allergen-challenged lung. Those cells that successfully migrated into the lung tissue were largely inhibited from the release of inflammatory mediators. These conclusions are supported by the inhibition of tissue mast cell degranulation, reduction in eosinophil numbers, reduction in airway cell differentiation to mucus secretion and in mucus plug formation. These observations suggest that HK-X successfully inhibits: increased integrin affinity; binding to ECM; intercolation of inflammatory cells into 3D matrix. Further, HK-X inhibited downstream events of inflammatory cell behavior such as degranulation and secretion of mediators, which support the synthesis, and secretion of ICAM and VCAM.

Additional experiments showed that parenteral administration of HK-X produced the same beneficial effects in the mouse model of chronic asthma. Interestingly, in this asthma model mast cell degranulation was significantly reduced by intranasal administration of HK-X during allergen challenge.

In the chronic asthma model HK-X was effective in removing eosinophils from the inflamed lung and reducing collagen deposition in the interstitial spaces. In this model HK-X was administered intranasally for up to 3 months. In control animals, chronic administration produced no pathological changes in the alveoli, bronchi or vessels.

In the chronic asthma model wherein inflammatory cells were resident prior to therapeutic intervention of HK-X, eosinophil numbers were reduced. Other potential downstream inflammatory effects of eosinophil infiltration were subsequently reduced. These observations are consistent with the notion that HK-X can interrupt or reverse VLA-6 and ECM interaction after successful integrin/ECM interaction, which include: increased integrin affinity; binding to ECM; formation of focal adhesions sites; and intercalation of inflammatory cells into 3D matrix.

### Detailed Materials and Methods for Example 7

1. Isolation of Cells

Human peripheral blood mononuclear and polymorphonuclear cells were isolated from peripheral blood obtained from normal donors. The blood was collected in heparin. The various cell types were isolated by centrifugation over Ficoll-Hypaque at 500×g for 60 min at room temperature. Each fraction was harvested, pooled separately and washed 1× in RPMI 1640 with antibiotics.

2. Culture and Treatment of Cells

10⁷ cells per ml of media were held at 37° C. for 30 min prior to the addition of stimulants in order to allow cells to reach a steady state within the phosphorylated protein pools. Then the following stimulants were added to each ml of cells:

| | |
|---|---|
| A. | 100 μL of vehicle (0.3% DMSO solution in media) |
| B. | 100 uL of HK-X contained 20 μg HK-X |
| C. | 100 uL of FMLP contained 0.1 μg FMLP |
| D. | 100 uL of IL-8 contained 0.1 μg IL-8 (recombinant |
| | human IL-8) |
| E. | 100 uL of HK-X contained 20 μg HK-X plus 100 μL of |
| | FMLP contained 0.1 μg FMLP |
| F. | 100 uL of HK-X contained 20 μg HK-X plus 100 μL of |
| | IL-8 contained 0.1 μg IL-8 |
| G. | cell culture media without and stimulants |

Cells were incubated for an additional 30 minutes at 37° C. in 5% CO₂.

3. Harvesting of Cells and SDS-PAGE Analysis

Cells were pelleted at 250×g for 5 min at room temperature. The supernatant was removed and 25 μL of 2× SDS-PAGE starting buffer added. The pellets were boiled for 15 min and centrifuges at 10,000×g for 5 min. Small samples were removed for gel electrophoresis on 12% acrylamide gels. In order to standardize the amount of cellular protein applied to each lane of the SDS-PAGE, the same number of cells were used for each treatment and approximately the same volume of sample applied to each lane.

4. Immunoblot Detection of Phosphoproteins

The proteins were transferred onto nylon membrane at 13 V for 30 min and subsequently blocked with 1% BSA for 12 hr. The antibody conjugated with HRP in 0.3% BSA was added for 60 min. Membranes were washed, fixed and photographed. The chemiluminescence patterns of phosphoproteins recognized by monoclonal anti-phosphotyrosine antibody were detected.

5. Data Analysis

The photographs were taken to a professional laboratory and a negative copy was made of each gel using very high contrast and low grain film. The subsequent photographs were scanned at 600 dpi and densitometric analysis performed using Image Pro Plus software, SPSS. Molecular weights were calculated for each band.

The pattern and distribution of protein kinases for peripheral blood polymorphonuclear cells was essentially the same as that for the mononuclear cells. The primary difference between the two cell types was that mononuclear cells were more metabolically active than the polymorphonuclear cells.

Using the densitometric analytical approach, the area under the peaks for each molecular weight species was calculated. Thus, the quantitative assessment of each kinase as a percent of the total kinase content was calculated. In addition the molecular weight of known kinases was compared to that calculated from the relative migration (RfP calculation in this experiment. Thus, the kinases in this study could be identified.

Table 3 illustrates the results of an experiment that shows the change in the distribution of protein kinases from human peripheral blood cells after exposure to HK-X compared to costimulatory exposure to (1) HK-X and (2) Ca5, TNFα, IL-4, IL-6, IL-10 or IL-13.

### Example 7

Signal Transduction Pathways of Various Chemokines and Cytokines

Because cytokines and chemokines utilize different signal pathways, we have presented an abbreviated table (Table 3) listing the chemokines/cytokines and their major pathways for signal transduction. Signal pathways that are unique to VLA-6 integrin also are presented. This table was not designed to be exhaustive or complete for every possible PK, cofactor or pathway.

TABLE 3
Summary of Major Signal Transduction Pathways of
Chemokines/Cytokines and FCε Receptors.

| Chemokine/Cytokine | Primary Pathway | Comments |
|---|---|---|
| fMLP | G-Proteins | ---LTB4 and PAF |
| C5a | G-Proteins | Use a different subset |
| IL-8 | G-Proteins | Of the G-Proteins |
| LTB4 | G-Proteins | Than fMLP, C5a, IL-8--- |
| Fc εR | G-Proteins | |
| IL-4 | ? | Ras implicated |
| IL-6 | JAK/TYK/Src | |
| IL-10 | JAK1/TYK2 | |
| IL-13 | JAK1/IRS/PI3 | Similar to IL-4 since uses |
| | | IL4R |
| TNFα | Numerous signals | |
| γIFN | JAK1/JAK2 | |
| VLA-6 | FAK | Activated by receptor cluster- |
| | | ing |
| | Ras | And Actin Polymerization and |
| | Raf | Other ligand systems |
| | MAPK/ERK | |

Leukocytes respond to a large number of chemoattractants and other pro-inflammatory mediators. Some mediators cause chemotaxis, activation of enzyme systems and release of pathologically significant mediators. The typical N-formyl peptides (the archetypal one—FMLP), activated complement fragment (C5a), leukotriene B4 (LTB4), platelet activating factor (PAF), and some chemotactic cytokines (such as IL-8) are well-recognized chemotactic and pro-inflammatory agents. These agents bind to G-protein-coupled receptors (GPCRs) with subsequent generation of multiple signal transduction mediated by protein kinase systems. The cascades resulting for the initial events are complex and interrelated, yet are responsible for the entire behavior of all nucleated cells. Programmed cell death (apoptosis), generation of immune responses, removal of self-recognizing T cells, and control of synthesis of extracellular matrices are just a few examples of the action of signal transduction pathways.

Protein kinases were identified by their capacity to transfer a phosphate group from a phosphate donor onto an acceptor amino acid located within a protein. Usually the y phosphate of ATP is the donor. The three major acceptor amino residues within proteins are tyrosine, serine and threonine. As of 1999, over 115 protein kinases have been identified and described in the literature.

The behavior of cells in response to stimulation with FMLP is well described in the literature (Prosnitz et al., Pharmacol. Ther. 74: 73-102, 1997). FMLP binding to phagocytes stimulates phosphorylation, which correlates with cellular functions. FMLP and other chemoattractants stimulate phosphatidylinositol 3-kinase (P13K) which in turn activates protein kinase (PKC). In neutrophils, FMLP binding initiates phosphorylation of an extracellular regulated kinase, (ERK-1) which belongs to a general family of kinases termed mitogen-activated protein kinases (MAP kinases). Some of the members of the MAP kinase family are: Raf-1 and Ras.

Members of the protein kinase families usually differ in molecular weight to such an extent that they can resolved one from another by SDS-PAGE technology. Further, phosphotyrosine proteins can be detected from the entire mass of intracellular proteins by monoclonal antibodies which recognize only the phosphotyrosine epitope (Ross et al., Nature (London)294: 654, 1981; Frackleton et al., Mol. Cell Biol. 3: 1343, 1983).

Changes in protein kinases mediated by the addition of HK-X to human peripheral blood mononuclear and polymorphonuclear cells were analyzed in order to elucidate the mechanism of action of the HK-X. HK-X was added alone and with the addition of FMLP or IL-8, which are known chemotactic and pro-inflammatory agents.

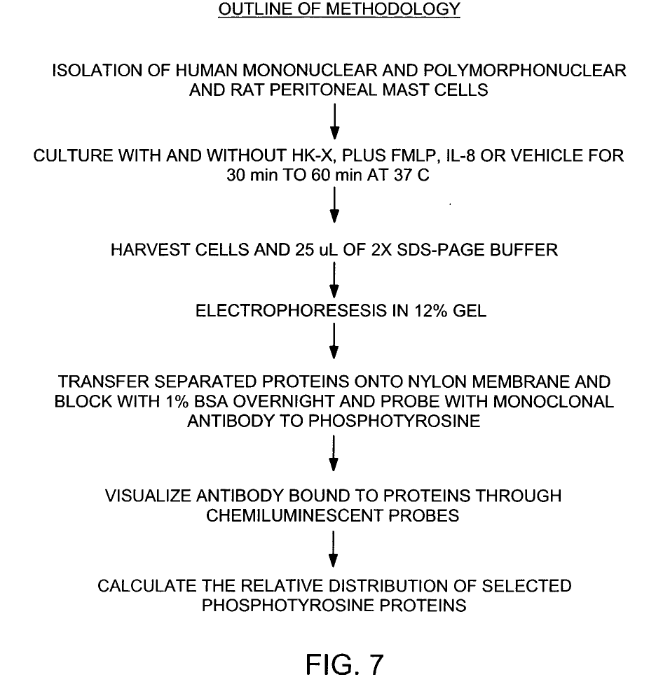

The protocol shown in FIG. 7 was used to obtain the information on the signaling proteins reported in Table 4.

TABLE 4
Distribution of Kinases After Costimulation With HK-X Plus Cytokines of Cell
Independent Pro-Inflammatory Compounds.

| | HK-X + | HK-X + | IL4 + | IL3 + | IL-6 + | TNFa + | C5a + | IL-10 + | HK-X |
|---|---|---|---|---|---|---|---|---|---|
| | FMLP | IL8 | HK-X | HK-X | HK-X | HK-X | HK-X | HK-X | Control |
| PI 3 [110 Kd] | 0.59 | 0.44 | 0.91 | 1.00 | 0.60 | 0.47 | 0.80 | 0.57 | 0.50 |
| PI3 [85 Kd] | 0.93 | 0.87 | 0.53 | 0.51 | 0.54 | 0.35 | 0.56 | 0.34 | 0.76 |
| Raf | 0.76 | 0.70 | 0.28 | 0.44 | 0.22 | 0.54 | 0.55 | 0.24 | 0.65 |
| Ras | 0.19 | 0.40 | 0.37 | 0.37 | 0.40 | 1.00 | 0.50 | 0.56 | 0.34 |
| Pp60 Src | 0.59 | 0.86 | 0.50 | 0.29 | 0.61 | 0.52 | 0.45 | 0.41 | 0.33 |
| ERK-1 | 0.40 | 0.63 | 0.45 | 0.45 | 0.43 | 0.55 | 0.50 | 0.65 | 0.46 |
| G-P α | 0.63 | 0.63 | 0.53 | 0.28 | 0.56 | 0.50 | 0.42 | 0.46 | 0.33 |
| G-P β | 0.33 | 0.37 | 0.53 | 0.52 | 0.52 | 0.53 | 0.50 | 0.50 | 0.44 |
| G-P γ | 0.10 | 0.16 | 0.37 | 0.25 | 0.46 | 0.45 | 0.56 | 0.52 | 0.52 |
| PLC γ | ND** | ND | 0.85 | 0.88 | 0.51 | 0.21 | 0.25 | 0.56 | 0.42 |

Table 4 shows the values determined by the following mathematical formula: [HK-X+Cytokine]/[HK-X alone+Cytokine alone].

If the values are less than 1.0, there is an effect where the cells exposed to both HK-X and costimulant provide a value which is less than the additive effects exhibited by each of the agents alone (i.e., HK-X alone or costimulant alone). If the value of the cells exposed to both HK-X and costimulant provide a value that is less than any of values shown by the agents alone (i.e. HK-X alone or costimulant alone), an inhibition was observed. The resulting ratio will be less than 0.5. For the HK-X Control, the HK-X value was divided by the sum of the value for the vehicle plus the fresh normal cells.

Not all of the signaling proteins and other transcriptional factors are shown in Table 4. Thus, the examples shown in Table 4 are not exhaustive and are not meant to be limiting to the scope of this invention.

In the case of integrin changes from low affinity to high affinity binding sites, organizational changes in actin, activation of FAK, focal adhesion formation and downstream merging with the MAPK pathway form critical sites for potential HK-X regulation.

Although these kinase studies were performed exclusively on peripheral blood mononuclear cells, the results presented can be extended to other cell types including mast cells and eosinophils.

In the cases of IL-4 and IL-13, 7 of the 10 kinases examined showed a nearly identical pattern of regulation. For example, the type and values of the interactions for PI3 (110 Kd), PI3 (85 Kd), ras, ERK, G-proteins β and γ, and PLC γ were similar. The IL-13 receptor has two components, one of which appears to be IL4Rα. Although IL-13 does not bind to the IL4Rα, this polypeptide chain appears to be an important component of the IL-13R. In contrast, the IL-13R complex may serve as an IL-4 receptor. It is tempting to speculate that HK-X mediates these highly similar downstream modulations of second messengers via its action on the common element, IL-4Rα.

The profiles for IL-6 and IL-10 are clear-cut. HK-X costimulation with IL-6 did not appreciably down or up regulate the tyrosine kinases. Raf did show a decrease; however, more downstream signal kinases (ERK) were largely unaffected. IL-6 and IL-10 mediate a wide range of effects on inflammatory cells, endothelial cells, lymphocyte interaction and activation. IL-6 has two pathways of responsiveness; in some cells both are operative and in some cell types, one is preferentially operative. Thus, interpreting the kinase response patterns for IL-6 observed with HK-X is difficult. However, having host target cells respond to IL-6 in a normal mode is consistent with our in vivo observations made on: (1) immune cell interactions in the generation of IgM and IgG PFCs, (2) B cell differentiation (immunoglobulin class change), (3) normal hematopoietic functioning (production of nucleated cells and release from marrow), and (4) normal production of thymic and marrow cells in HK-X treated mice.

Other parameters of health and homeostasis in our studies suggest that, to a large extent, regulation and expression of IL-6 were not significantly compromised. IL-10 activities in our model are much less susceptible to clear interpretation. IL-10 mediates a host of pleiotrophic activities and some of its actions appear contradictory.

The behavior of TNFα receptor differs from other of the cytokines in that receptor clustering is the key signaling event with subsequent downstream signaling which involves TRADD, FADD, and RIP. Subsequently, many competing processes become operative including of both simultaneous generation of self-destructive radicals, on one hand as well as protective pathways on the other. If increased Ras could stimulate the Jun pathway, this should facilitate activation of NFκB. Exactly what role Ras plays in the subsequent behavior of TNFα-treated cells is beyond the scope of this research.

HK-X binds to and antagonizes the FPR in concert with chemokines and cytokines binding to their respective receptors. Simultaneously, HK-X binds to the alpha 6 subunit of VLA-6. This dual receptor binding under costimulatory conditions initiates a number of significant changes in the FPR, chemokines and cytokines, and integrin-directed pathways. These pathways are principally the FAK-Ras-Raf-MEK pathway as well as the Ras-Raf-MAPKK-ERK pathway. These pathways are not mutually exclusive and enjoin cross talk at various points in their changed signals and calcium flux. Direct binding of HK-X to the alpha 6 subunit of integrin VLA-6 may have as yet unexplained effects on the VLA-6 molecule. Specifically, HK-X may diminish integrin clustering, become activated, and send downstream kinase signals to pro-inflammatory cells and other integrins.

A strong absence of binding and migration of pro-inflammatory cells to the site of inflammation (including reduction in the binding of ECMs) and reduction in collagen deposition is consistent with our in vivo evidence of integrin receptor antagonism affected by HK-X. On the other hand, present data suggests that HK-X down-regulates the pro-inflammatory response to inflammatory molecules remaining resident in the tissue surrounding and at the site of inflammation through its antagonism of the FPR. This is evidenced in vivo by the strong clearing of cellular infiltrate, reduction of mucus plugs and reduction of ICAM and VCAM.

Considered together, HK-X's simultaneous involvement with two important regulatory receptors—under specific conditions of co-stimulatory challenge by one or more major inflammation mediators—provides a uniquely powerful tool for therapeutic development in humans. The fact that fMLPP exhibits no discernable toxicity is promising for therapeutic treatment of the variety of indications resulting from an alpha 6 subunit containing integrin-mediated response.

Other “alpha 6 subunit containing integrin-mediated signal transduction pathway modification agents” can be determined by routine experimentation using an affinity purification procedure. A suspected “alpha 6 subunit containing integrin-mediated signal transduction pathway modification agent” is attached to an affinity column such as a Sepharose® column. Alpha 6 integrin subunits are labelled with ³⁵S-methionine and are passed over the suspected agent substituted Sepharose® on the column. Alpha 6 integrin subunits bound to the Sepharose® can be specifically recovered by eluting in the presence of 1 mg suspected agent(competitor) using pH 2.5 acid conditions.

Alternatively, other agents that can complex with an alpha 6 subunit containing integrin, e.g., VLA-6, and modify the signal transduction pathway of an alpha 6 subunit containing integrin, can be determined by routine experimentation using an affinity purification procedure. An affinity column can be made by attaching alpha 6 integrin subunit to a column resin such as a Sepharose® resin. Peptides, proteins or other compounds can be passed over the alpha 6 integrin subunit substituted Sepharose® on the column. Agents that bind to the alpha 6 substituted Sepharose® can be specifically recovered by eluting in the presence of excess alpha 6 subunit protein (competitor) or by using acidic conditions (pH 2.5). Eluted agents can be isolated from an SDS-PAGE gel and chemical and spectral analysis can then be performed to identify the alpha 6 subunit interacting agent.

The present invention has been described in detail including the preferred embodiments thereof. However, it will be appreciated that those skilled in the art may make modifications and improvements within the spirit and scope of the invention as set forth in the claims.

What is claimed is:

1. A method for treating an indication resulting from an alpha 6 subunit containing integrin-mediated pathological condition in a mammal, the method comprising administering to the mammal a treatment effective amount of an alpha 6 subunit containing integrin-mediated signal transduction pathway modification agent.

2. The method of claim 1, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr, wherein said peptide is capable binding with an α₆ integrin subunit.

3. The method of claim 1, wherein the cell is contacted with an additional active ingredient along with said peptide, said active ingredient being selected from the group consisting of anti-leukotrienes, beta₂ antagonists and corticosteroids.

4. The method of claim 1, wherein said condition is an inflammation.

5. The method of claim 3, wherein said inflammation is mediated by a pro-inflammatory agent selected from the group consisting of cytokines, chemokines, chemotaxins and mitogens.

6. The method of claim 4, wherein said pro-inflammatory agent selected from the group consisting of fMLP, activated complement fragment, leukotriene B4, platelet activating factor, IL-4, IL-6, IL-8, IL-10, IL-13 and TNFα.

7. The method of claim 1, wherein said alpha 6 subunit containing integrin-mediated pathological condition is cell mestastasis.

8. The method of claim 1, wherein said alpha 6 subunit containing integrin-mediated pathological condition is coronary heart disease.

9. The method of claim 1, wherein said peptide is f-Met-Leu-Phe-Phe.

10. A method for modulating the function of an α₆ subunit containing integrin, said method comprising contacting a cell having the α₆ subunit containing integrin with an effective function modulating amount of an alpha 6 subunit containing integrin-mediated signal transduction pathway modification agent, thereby modulating the integrin signal transaction pathway of said integrin.

11. The method of claim 10, wherein said an alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

12. The method of claim 10, wherein said cell is a selected from the group consisting of peripheral blood mononuclear cells, peripheral blood polymorphonuclear cells, lymphocytes, granulocytes, eosinophils, basophils, dendritic cells, astrocytes, macrophages, activated T-cells and mast cells.

13. A cell surface complex comprising a cell surface α₆ integrin subunit and an alpha 6 subunit containing integrin-mediated signal transduction pathway modification agent.

14. The cell surface complex of claim 13, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

15. A method for modulating an alpha 6 subunit containing integrin-mediated response, said method comprising forming a complex of a cell surface α₆ integrin subunit and an alpha 6 subunit containing integrin-mediated signal transduction pathway modification agent.

16. The method of claim 15, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

17. A cell surface receptor complex comprising:
an α₆ integrin subunit and an alpha 6 subunit containing integrin-mediated signal transduction pathway modification agent,
wherein compared to cells not contacted by the candidate integrin-mediated signal transduction pathway modification agent there is a change in the amount of PI3, Raf, Ras, Src, Erk-1, PLC γ, G-protein α, G-protein β or G-protein γ kinases.

18. A cell surface complex comprising the VLA-6 integrin receptor and an alpha 6 subunit containing integrin-mediated signal transduction pathway modification agent.

19. The cell surface complex of claim 18, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

20. A method for modulating an VLA-6 integrin-mediated response, said method comprising forming a complex of the VLA-6 integrin receptor and an alpha 6 subunit containing integrin-mediated signal transduction pathway modification agent.

21. The method of claim 20, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

22. A cell surface receptor complex comprising an α₆ integrin subunit and an alpha 6 subunit containing integrin-mediated signal transduction pathway modification agent, wherein compared to cells not contacted by the candidate integrin-mediated signal transduction pathway modification agent there is a change in the amount of PI3, Raf, Ras, Src, Erk-1, PLC γ, G-protein α, G-protein β or G-protein γ kinases.

23. A cell surface complex comprising the VLA-6 integrin receptor and an alpha 6 subunit containing integrin-mediated signal transduction pathway modification agent.

24. The cell surface complex of claim 23, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

25. A method for modulating an integrin-mediated response, said method comprising forming a complex of the VLA-6 integrin receptor and an alpha 6 subunit containing integrin-mediated signal transduction pathway modification agent.

26. The method of claim 25, wherein said alpha 6 subunit containing integrin-mediated signal transduction pathway modification agent is a peptide having the formula f-Met-Leu-X where X is selected from the group consisting of Tyr, Tyr-Phe, Phe-Phe and Phe-Tyr.

27. A method for identifying an alpha 6 subunit containing integrin-mediated signal transduction pathway modification agent, the method comprising the steps of:
attaching an alpha 6 subunit to an affinity column;
passing a solution containing a suspected alpha 6 subunit containing integrin-mediated signal transduction pathway modification agent over the affinity column substituted with an alpha 6 subunit to bind the suspected agent;
recovering bound agent by eluting in the presence of excess alpha 6 subunit; and
identifying the eluted bound agent.
